(19) United States
(12) Patent Application Publication
(10) Pub. No.: US 2026/0265794 A1
(43) Pub. Date: Sep. 10, 2026

(54) A PROCESS FOR PREPARATION OF L-GLUFOSINATE OR SALTS THEREOF

(71) Applicants: UPL LIMITED, Mumbai (IN); INSTITUTE OF CHEMICAL TECHNOLOGY, Mumbai (IN)

(72) Inventors: Prashant Vasant Kini, Mumbai (IN); Chandrasekhar Dayal Mudaliar, Mumbai (IN); Ashishkumar Ravindra Mishra, Mumbai (IN); Annamma Anil Odaneth, Mumbai (IN); Rasika Vivek Tupe, Mumbai (IN)

(73) Assignees: UPL LIMITED, Mumbai (IN); INSTITUTE OF CHEMICAL TECHNOLOGY, Mumbai (IN)

(21) Appl. No.: 19/164,051

(22) PCT Filed: Mar. 27, 2024
(86) PCT No.: PCT/IN2024/050324

§ 371 Date: Sep. 10, 2025

(30) Foreign Application Priority Data
Mar. 27, 2023 (IN) 202321022181

(51) Int. Cl. C12P 13/04; A01N 57/20; A01P 13/00

(52) U.S. Cl. CPC C12P 13/04; A01N 57/20; A01P 13/00

(57) ABSTRACT

The present invention relates to a green and cost-effective biocatalytic process for preparing L-glufosinate, esters, or salts thereof. The biocatalytic process comprises converting 2-oxo-4-(hydroxymethylphosphinyl)butyric acid to L-glufosinate, 5 esters, or salts thereof, under the action of a biocatalyst.

### FIELD OF THE INVENTION

The present invention relates to a green and cost-effective process for preparing L-glufosinate, esters, or salts thereof. More particularly, the present invention relates to a biocatalytic process for preparing L-glufosinate, esters, or salts thereof.

### BACKGROUND OF THE INVENTION

Glufosinate is a non-selective herbicide belonging to the group of organophosphate herbicides; and has been widely used around the world. It is generally used in the form of ammonium salt for total vegetation control and to control growth of weeds and grasses. Glufosinate is used as a racemic mixture of L-glufosinate & D-glufosinate. However, it is well known that L-glufosinate i.e., (S)-2-amino-4-(hydroxy(methyl)phosphoryl)butanoic acid) is much more potent than D-glufosinate. The L-isomer of glufosinate is a structural analogue of glutamate and, therefore, is a competitive inhibitor of the enzyme glutamine synthetase (GS) of bacteria and plants. The L-enantiomer of glufosinate acts by inhibition of glutamine synthetase thereby causing accumulation of toxic levels of ammonium ion and indirectly stopping photosynthesis.

Mainly there are three methods known in prior art for preparing optically pure L-glufosinate, namely by asymmetric chemical synthesis, by chiral separation and by biocatalytic method.

The asymmetric chemical synthesis method is based on the synthesis of optically pure L-glufosinate, which is more common in laboratory research. One such process is provided in the Journal of Organic Chemistry, 1991, 56: 1783-1788, however, such processes involve many steps and provide low yield, and the asymmetric synthetic reagents used are mostly expensive, resulting in high production cost, which is not feasible for large-scale production of L-glufosinate.

In the chiral separation method, resolution of racemic glufosinate or their salts is carried out using chiral resolution reagent. Patent publication WO1995023805 provides a process wherein racemic glufosinate or its salt is resolved to get L-glufosinate using chiral bases like quinine, cinchonine, cinchonidine or brucine. The major disadvantage of this process is use of expensive chiral resolution reagent which impacts overall cost of process thereby making it difficult to use this process at industrial scale.

Lastly, the biocatalytic synthesis method is considered to be the perfect “green” technique. This method has many advantages such as mild reaction conditions, low toxicity, high stereoselectivity, and the production of eco-friendly waste; and is suitable for industrial scale production of optically pure L-glufosinate. The biocatalytic synthesis method involves use of isolated enzymes or whole cells (such as bacteria, fungi, microalgae and plants) as catalysts in organic reactions. Most of the publications on biocatalytic synthesis method for producing L-glufosinate or its salts focus on use of isolated enzymes obtained by the overexpression of enzymes in genetically engineered microorganisms. However, identification of gene, genetic modification of microorganism to provide desired enzymes, isolation of such enzymes, makes this technique cumbersome and expensive. Also, such isolation of enzymes requires special procedure and resources.

Therefore, the inventors of the present invention have developed a biocatalytic process for obtaining L-glufosinate, esters, or salts thereof, using a biocatalyst, thereby gaining all the advantages of biocatalytic synthesis and still avoiding the aforementioned hindrances of such processes.

### OBJECTIVES OF THE INVENTION

A primary objective of the present invention is to provide a green process for obtaining L-glufosinate, esters, or salts thereof.

Another objective of the present invention is to provide a biocatalytic method for preparing L-glufosinate, esters, or salts thereof, having high yield and purity.

Another objective of the present invention is to provide a simple, inexpensive and efficient biocatalytic process for obtaining L-glufosinate, esters, or salts thereof.

Yet another objective of the present invention is to provide an environment-friendly process for obtaining L-glufosinate, esters, or salts thereof.

### SUMMARY OF THE INVENTION

According to an aspect of the present invention, there is provided a biocatalytic process for preparation of L-glufosinate, esters, or salts thereof.

According to an aspect of the present invention, there is provided a biocatalytic process for preparing L-glufosinate, esters, or salts thereof, the process comprising converting 2-oxo-4-(hydroxymethylphosphinyl)butyric acid to L-glufosinate, esters, or salts thereof, using a biocatalyst derived from a micro-organism of Amycolatopsis sp.

According to an aspect of the present invention, there is provided a biocatalytic process for preparing L-glufosinate, esters, or salts thereof, the process comprising converting 2-oxo-4-(hydroxymethylphosphinyl)butyric acid to L-glufosinate, esters, or salts thereof using a biocatalyst derived from a micro-organism of Amycolatopsis sp. deposited as ATCC 39116.

According to another aspect of the present invention, there is provided a biocatalytic process for preparing L-glufosinate, esters or salts thereof, the process comprising converting 2-oxo-4-(hydroxymethylphosphinyl)butyric acid to L-glufosinate, esters or salts thereof using a biocatalyst derived from a micro-organism of Amycolatopsis sp., in presence of an amine donor.

According to another aspect of the present invention, there is provided a biocatalytic process for preparing L-glufosinate, esters, or salts thereof, the process comprising converting 2-oxo-4-(hydroxymethylphosphinyl)butyric acid to L-glufosinate, esters, or salts thereof using a biocatalyst derived from a micro-organism of Amycolatopsis sp., in presence of an amine donor and optionally a cofactor.

Yet another aspect provides an L-glufosinate, esters, or salts thereof obtainable by a process comprising converting 2-oxo-4-(hydroxymethylphosphinyl)butyric acid to L-glufosinate, esters, or salts thereof, using a biocatalyst derived from a micro-organism of Amycolatopsis sp.

In another aspect, there is provided an agrochemical composition comprising L-glufosinate, esters, or salts thereof prepared by the biocatalytic process of the present invention.

### BRIEF DESCRIPTION OF THE DRAWINGS

FIG. 1 illustrates the high-performance liquid chromatography (HPLC) profile of reactants and products on 14^{th} day according to Example 4 of the present invention.

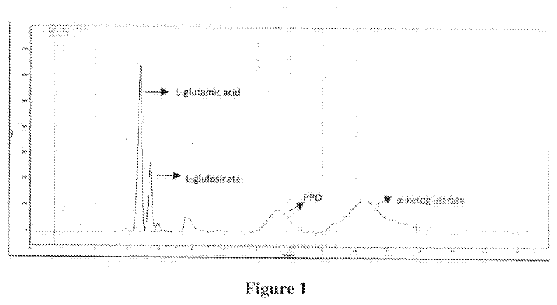

FIG. 2 illustrates chiral HPLC analysis of the product.

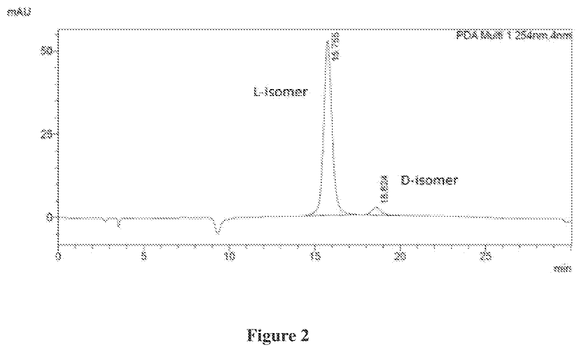

### DETAILED DESCRIPTION OF THE INVENTION

Those skilled in art will be aware that invention described herein is subject to variations and modifications other than those specifically described. It is to be understood that the invention described herein includes all such variations and modifications. The invention also includes all such steps, features, compositions and methods referred to or indicated in this specification, individually or collectively, and any and all combinations of any two or more said steps or features. For convenience, before providing further description of the present invention, certain terms employed in the specification, examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances. The terms used herein are defined as follows.

As used in the specification and the claims, the singular forms “a”, “an”, and “the” include plural referents unless the context clearly dictates otherwise. The present disclosure is not to be limited in scope by the specific embodiments described herein, which are intended for the purposes of exemplification only.

The term “room temperature” unless stated otherwise, essentially means temperature in range of about 20° C. to 35° C.

The term “purity” means purity as determined by HPLC.

The term “about” shall be interpreted to mean “approximately” or “reasonably close to” and any statistically insignificant variations therefrom. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or ±5% of the stated value. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

As used herein, the terms “comprising”, “including”, “having”, “containing”, “involving”, and the like are to be understood to be open-ended, i.e., to mean including but not limited to.

The terms “preferred” and “preferably” refer to embodiments of the invention that may afford certain benefits, under certain circumstances. In an embodiment, the aspects and embodiments described herein shall also be interpreted to replace the clause “comprising” with either “consisting of” or with “consisting essentially of” or with “consisting substantially of”.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

The term “glufosinate” refers to an isomeric mixture of L-glufosinate and D-glufosinate.

As used herein, the term “L-glufosinate” includes the L-isomer of glufosinate, a salt and derivatives thereof. The term may also refer to L-glufosinate, wherein the content of L-glufosinate is 70% or greater, preferably 80% or greater, and more preferably 90% or greater. Typically, the ratio of L-glufosinate:D-glufosinate can be in the range from about 90:10 to about 100:0.

“L-glufosinate” is also known as L-phosphinothricin or (S)-2-amino-4-(hydroxy(methyl)phosphoryl)butanoic acid. The term can generically refer to any form of L-glufosinate such as solvates, hydrates, anhydrous form, polymorph forms, pseudo polymorph forms, amorphous form or mixture thereof, and derivatives such as esters; and salts. The term “L-glufosinate” shall be interpreted to mean L-glufosinate or its salts. The salts of L-glufosinate such as monosodium salt, disodium salt, monopotassium salt, dipotassium salt, calcium salt, ammonium salt, —NH₃(CH₃)⁺ salt, —NH₂(CH₃)₂⁺ salt, —NH(CH₃)₃⁺ salt, —NH(CH₃)₂(C₂H₄OH)⁺ salt, and —NH₂(CH₃)(C₂H₄OH)⁺ salt are included in the definition. The agronomically acceptable salts include L-glufosinate-ammonium, L-glufosinate-sodium, and L-glufosinate-potassium.

The term “% enantiomeric excess” or “% ee” means the enantiomeric purity of a sample, that is, the percentage of one enantiomer that exceeds the other enantiomer in the sample. For example, the enantiomeric excess of L-glufosinate is the percentage of L-glufosinate that exceeds D-glufosinate in the glufosinate.

It should be noted that, as used herein, “derivative” or “analogue” of a molecule refers to a portion derived from or a modified version of the molecule.

As used herein, “biocatalyst” refers to a whole cell, particularly, a microorganism, comprising a natural catalyst or one or more enzymes to perform chemical transformations on organic compounds; or isolated enzymes, partially purified enzymes, cell-free extracts or crude cell extract liquid/powder/immobilized or fixed form, permeabilized cells, whole cells, whole fermentation broths, lyophilized cells, from such microorganisms.

As used herein, “Amycolatopsis sp. ATCC 39116” refers to the organism of Amycolatopsis sp. deposited with the American Type Culture Collection (ATCC) patent depository in the U.S.A having the collection no. 39116.

As used herein “transaminase/amino acid transferase” refers to an enzyme, a catalytically active portion, derivative or analogue thereof, which catalyses the interconversion of amino acids and oxoacids by transfer of amino groups.

As used herein “Amino acid dehydrogenase” refers to an enzyme belonging to the group of oxidoreductases that catalyses oxidative deamination of primary amines by reducing an electron acceptor, usually NAD/NADP to form aldehyde and ammonia.

As used herein “Aldo-keto reductases” refers to a superfamily of NAD(P)H-dependent oxidoreductases which reduce aldehydes and ketones to their respective primary and secondary alcohols.

The term “pre-incubation” as used herein means to incubate (microorganisms/whole-cell catalyst) for 1 to 20 hours prior to a biocatalytic process according to present invention.

The term “2-oxo-4-(hydroxymethylphosphinyl)butyric acid” is also referred to as “PPO” or “substrate”, and both the terms are used interchangeably throughout the description.

The term “% Assimilation” as used herein means percentage of substrate consumed by biocatalyst in relation to the total substrate added at the start of reaction.

The term “% Conversion” as used herein means percentage of total product formed by biocatalyst in relation to the total substrate added at the start of reaction.

According to an aspect of the present invention, there is provided a biocatalytic process for obtaining L-glufosinate, esters, or salts thereof.

According to an aspect of the present invention, there is provided a process for preparing L-glufosinate, esters, or salts thereof, the process comprising: converting 2-oxo-4-(hydroxymethylphosphinyl)butyric acid to L-glufosinate, esters, or salts thereof using a biocatalyst derived from a micro-organism of Amycolatopsis sp.

In another embodiment, the micro-organism of Amycolatopsis sp. is deposited as ATCC 39116.

In an embodiment, the substrate 2-oxo-4-(hydroxymethylphosphinyl)butyric acid is prepared by conventionally known processes such as an enzymatic process or a chemical process.

In an embodiment, the process is carried out at a substrate concentration of about 20 g/L to about 400 g/L. In an embodiment, the process is carried out at a substrate concentrations of about 80 g/L to about 400 g/L. In another embodiment, the process is carried out at a substrate concentrations of about 100 g/L to about 300 g/L.

In an embodiment, total amount of substrate is added at the beginning of the reaction. In another embodiment, the substrate is metered in batchwise during the reaction.

In an embodiment, the micro-organism of Amycolatopsis sp., may produce multiple enzymes, preferably one or more enzymes. According to an embodiment, the Amycolatopsis sp., produces at least one enzyme capable of catalysing the conversion of a substrate to L-glufosinate, esters, or salts thereof.

According to an embodiment, the biocatalyst comprises one or more enzymes produced by the micro-organism of Amycolatopsis sp. selected from aminotransferase and/or amino-acid dehydrogenase and/or aldo-keto reductases.

According to an embodiment, the biocatalyst comprises at least one aminotransferase and/or at least one amino-acid dehydrogenase and/or at least one aldo-keto reductases, or combinations thereof. The aminotransferase enzymes belong to class of transferase, which catalyses the transfer of an amino group from an amino donor to a prochiral acceptor ketone to obtain a chiral amine and a corresponding ketone or α-keto acids. The microorganism of Amycolatopsis sp. further produces additional transferases other than transaminases. The amino acid dehydrogenase enzyme refers to an enzyme belonging to the group of oxidoreductases that catalyses oxidative deamination of primary amines by reducing an electron acceptor, usually NAD/NADP to form aldehyde and ammonia. The microorganism of Amycolatopsis sp. further produces additional dehydrogenases other than amino-acid dehydrogenases. The aldo-keto reductase enzymes refers to a superfamily of NAD(P)H-dependent oxidoreductases which reduce carbonyl substrates.

According to an embodiment, the biocatalyst is selected from, but not limited to, isolated enzymes; partially purified enzymes; cell-free extract or crude cell extract in liquid, powder, or immobilized/fixed form; permeabilized cells, whole cells, whole fermentation broths, lyophilized cells, or combinations thereof.

In a preferred embodiment, the biocatalyst comprises whole cells, whole fermentation broths, permeabilized cells, or lyophilized cells. In a more preferred embodiment, the biocatalyst comprises whole cells or whole fermentation broths.

In an embodiment, the Amycolatopsis sp., is enriched and cultured by culture media, and then the cells are centrifuged, collected and used as a whole-cell catalyst for biocatalytic process as provided in the present invention. The process of culturing the microorganism and selection of culture media is carried out using conventional processes known to a person skilled in the art.

In an embodiment, the amount of Amycolatopsis sp. in terms of wet cell weight is in a range from about 50 g/L to about 600 g/L.

In an embodiment, the amount of Amycolatopsis sp. in terms of wet cell weight is about 100 g/L to about 200 g/L. In an embodiment, the amount of Amycolatopsis sp. in terms of wet cell weight is about 200 g/L to about 300 g/L. In an embodiment, the amount of Amycolatopsis sp. in terms of wet cell weight is about 300 g/L to about 400 g/L. In an embodiment, the amount of Amycolatopsis sp. in terms of wet cell weight is about 400 g/L to about 500 g/L. In an embodiment, the amount of Amycolatopsis sp. in terms of wet cell weight is about 500 g/L to about 600 g/L.

In an embodiment, the process of converting 2-oxo-4-(hydroxymethylphosphinyl)butyric acid (PPO) to L-glufosinate, esters, or salts thereof, involves specific amination of PPO to L-glufosinate using an amine group from one or more amine donors.

In an embodiment, 2-oxo-4-(hydroxymethylphosphinyl)butyric acid is converted to L-glufosinate, esters, or salts thereof using a biocatalyst derived from a microorganism of Amycolatopsis sp. and in presence of an amine donor. The process of the present invention is carried out in presence of an amine donor.

According to another aspect of the present invention, there is provided a process for preparing L-glufosinate, esters, or salts thereof, wherein 2-oxo-4-(hydroxymethylphosphinyl) butyric acid is converted to L-glufosinate, esters, or salts thereof using a biocatalyst derived from a micro-organism of Amycolatopsis sp., in presence of an amine donor.

In an embodiment, the amine donor is selected from, but not limited to, L-aspartate or racemic aspartate or salts thereof, L-glutamate or racemic glutamate or salts thereof, L-alanine or racemic alanine or salts thereof, L-phenylalanine or racemic phenylalanine or salts thereof, L-glycine or racemic glycine or salts thereof, L-lysine or racemic lysine or salts thereof, L-valine or racemic valine or salts thereof, L-serine or racemic serine or salts thereof, L-glutamine or racemic glutamine or salts thereof; inorganic ammonia source, ammonia; organic amine such as isopropylamine, sec-butylamine, ethanolamine, 2-aminobutyric acid or salts thereof, and diaminoproprionic acid or salts thereof.

In a preferred embodiment, the amine donor is selected from L-glutamate or racemic glutamate or salts thereof. In another preferred embodiment, the amine donor is monosodium L-glutamate.

In another embodiment, the process is carried out in presence of amine donor present in a concentration of about 100 g/L to about 800 g/L in the reaction solution. In a preferred embodiment, the process is carried out in presence of amine donor present in a concentration of about 200 g/L to about 600 g/L in the reaction solution. In a preferred embodiment, the process is carried out in presence of amine donor present in a concentration of about 200 g/L in the reaction solution. In another preferred embodiment, the process is carried out in presence of amine donor present in a concentration of about 500 g/L in the reaction solution.

In an embodiment, the Amycolatopsis sp. is pre-incubated prior to contact with the substrate. In another embodiment, the Amycolatopsis sp. is pre-incubated for about 1 hr to about 20 hours with the amine donor.

In an embodiment, the process for preparation of L-glufosinate, esters, or salts thereof is optionally carried out in presence of a cofactor. In an embodiment, the process for preparation of L-glufosinate, esters, or salts thereof is carried out in presence of a cofactor. The cofactor is pyridoxal 5′ phosphate.

In an embodiment, the cofactor is either added externally or produced by Amycolatopsis sp. In a preferred embodiment, the cofactor is externally added. In another preferred embodiment, the cofactor is produced by Amycolatopsis sp.

In another embodiment, the process of present invention is carried out without external addition of a cofactor. This means that the organisms themselves contain or are capable of generating cofactor suitable for converting the substrate to L-glufosinate, esters, or salts thereof.

In an embodiment, the step of converting 2-oxo-4-(hydroxymethylphosphinyl)butyric acid to L-glufosinate, esters, or salts thereof, comprises treating 2-oxo-4-(hydroxymethylphosphinyl)butyric acid with an alkali solution.

In an embodiment, 2-oxo-4-(hydroxymethylphosphinyl)butyric acid is treated with an alkali solution before addition of biocatalyst, amine donor and optionally cofactor.

In an embodiment, 2-oxo-4-(hydroxymethylphosphinyl)butyric acid is first treated with an alkali solution followed by adding all the reagents.

In an embodiment, 2-oxo-4-(hydroxymethylphosphinyl)butyric acid is first treated with an alkali solution followed by adding biocatalyst, amine donor and optionally cofactor.

In another embodiment, 2-oxo-4-(hydroxymethylphosphinyl)butyric acid and amine donor are first treated with an alkali solution followed by adding biocatalyst and optionally cofactor.

In an embodiment, the treatment of 2-oxo-4-(hydroxymethylphosphinyl)butyric acid with an alkali solution comprises neutralisation of 2-oxo-4-(hydroxymethylphosphinyl)butyric acid.

In another embodiment, all the reagents along with substrate are added to the reaction medium followed by treatment with an alkali solution.

In another embodiment, 2-oxo-4-(hydroxymethylphosphinyl)butyric acid, biocatalyst, amine donor and optionally cofactor are added to the reaction medium and then the reaction medium is treated with an alkali solution. The reaction medium comprises water, preferably DM water.

In an embodiment, the alkali solution comprises, but not limited to, sodium hydroxide, sodium carbonate, sodium bicarbonate, potassium hydroxide, potassium carbonate, potassium bicarbonate, ammonium hydroxide, ammonium carbonate, ammonium bicarbonate, or combinations thereof.

In an embodiment, the alkali solution is aqueous solution of an alkali. In a preferred embodiment, the alkali solution is aqueous sodium hydroxide. In another preferred embodiment, the alkali solution is aqueous sodium carbonate. In a yet another preferred embodiment, the alkali solution is aqueous ammonium carbonate.

In an embodiment, the process of converting 2-oxo-4-(hydroxymethylphosphinyl)butyric acid to L-glufosinate, esters, or salts thereof, is carried out at pH ranging from about 6 to about 9. In an embodiment, optionally the pH is adjusted with a buffer solution. The buffer solution is selected from acetate buffer, phosphate buffer or Tris-HCl buffer.

In another embodiment, the biocatalytic process according to present invention is carried out at a temperature ranging from about 20° C. to about 60° C.

In an embodiment, there is provided a process for preparing L-glufosinate, esters, or salts thereof, the process comprising:
converting 2-oxo-4-(hydroxymethylphosphinyl) butyric acid to L-glufosinate, esters, or salts thereof using a biocatalyst derived from a micro-organism of Amycolatopsis sp., in presence of monosodium L-glutamate and optionally pyridoxal 5′ phosphate.

In an embodiment, there is provided a process for preparing L-glufosinate, esters, or salts thereof, the process comprising:
converting 2-oxo-4-(hydroxymethylphosphinyl) butyric acid to L-glufosinate, esters, or salts thereof using a biocatalyst derived from a micro-organism of Amycolatopsis sp., in presence of monosodium L-glutamate and pyridoxal 5′ phosphate.

In an embodiment, prior to contacting with the substrate, the Amycolatopsis sp. is pre-incubated for about 1 hr to about 20 hours with the amine donor and the cofactor. In another embodiment, the Amycolatopsis sp. is not pre-incubated prior to contacting with the substrate; and is directly used.

In an embodiment, the process of the present invention optionally comprises batchwise addition of the substrate 2-oxo-4-(hydroxymethylphosphinyl)butyric acid.

In another embodiment, the Amycolatopsis sp. is capable of producing enzyme(s) required for said conversion, multiple times and hence, can be reused or recycled.

A single batch of the Amycolatopsis sp. can be reused or recycled at least about 5 times for biocatalytic process according to present invention. In another embodiment, the biocatalyst is recycled at least about 5 times.

In another embodiment, a single batch of Amycolatopsis sp. can be reused or recycled about 5 to about 15 times for the biocatalytic process according to the present invention. In another embodiment, a single batch of the Amycolatopsis sp. can be recycled for about 5 to about 8 times for the biocatalytic process according to the present invention.

The inventors of present invention found that the micro-organisms used remain catalytically competent for several days and can be reused or recycled multiple times to catalyse the biotransformation of the substrate to L-glufosinate, esters, or salts thereof.

In an embodiment, the micro-organisms used in the present process are easily separated from the reaction system by conventionally known processes such as centrifugation and filtration. The separation technique comprises centrifugation, filtration, and the likes.

Thus, in an embodiment, the biocatalyst used in the process described in the present invention can be recovered and/or recycled by conventional processes such as centrifugation and/or filtration.

In an embodiment, the general scheme of the biocatalytic process according to the present invention can be represented as in scheme (I):

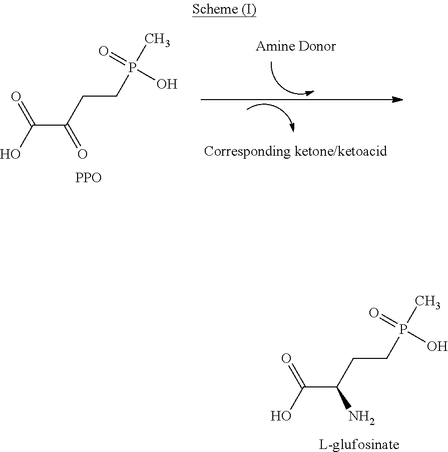

In an embodiment, the present invention provides a biocatalytic process for preparing L-glufosinate, esters, or salts thereof, the process comprising: converting 2-oxo-4-(hydroxymethylphosphinyl)butyric acid to L-glufosinate, esters, or salts thereof and a corresponding ketone or α-keto acids, using a biocatalyst derived from a micro-organism of Amycolatopsis sp. and in presence of an amine donor.

In an embodiment, the present invention provides a biocatalytic process for preparing L-glufosinate, esters, or salts thereof, wherein 2-oxo-4-(hydroxymethylphosphinyl)butyric acid is converted to L-glufosinate, esters, or salts thereof and a corresponding ketone or α-keto acids, using a biocatalyst derived from a micro-organism of Amycolatopsis sp., in presence of an amine donor and optionally a cofactor.

In an embodiment, the present invention provides a biocatalytic process for preparing L-glufosinate, esters, or salts thereof, wherein 2-oxo-4-(hydroxymethylphosphinyl)butyric acid is converted to L-glufosinate, esters, or salts thereof and a corresponding ketone or α-keto acids, using a biocatalyst derived from a micro-organism of Amycolatopsis sp., in presence of an amine donor and a cofactor.

In an embodiment, the corresponding ketone or α-keto acids obtained in the biocatalytic process can be enzymatically converted to an amine donor by conventionally known processes and used in the biocatalytic process of the present invention.

In an embodiment, the biocatalytic process according to the present invention can be represented as in scheme (II):

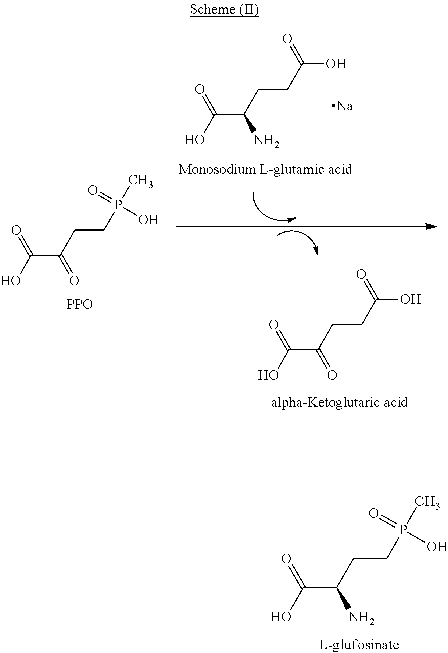

In an embodiment, the amine donor is monosodium L-glutamate. In an embodiment, the corresponding keto compound formed is α-ketoglutaric acid.

In an embodiment, the enantiomeric excess of the L-glufosinate, esters, or salts thereof obtained, having L-form may be, for example, 10% ee or more, 20% ee or more, 30% ee or more, 40% ee or more, 50% ee or more, 60% ee or more, 70% ee or more, 80% ee or more, 90% ee or more, 91% ee or more, 92% ee or more, 93% ee or more, 94% ee or more, 95% ee or more, 96% ee or more, 97% ee or more, 98% ee or more, or 99% ee or more.

In an embodiment, the % assimilation of 2-oxo-4-(hydroxymethylphosphinyl)butyric acid is at least 30%. In an embodiment, the % assimilation of 2-oxo-4-(hydroxymethylphosphinyl)butyric acid is at least 40%. In an embodiment, the % assimilation of 2-oxo-4-(hydroxymethylphosphinyl)butyric acid is at least 50%.

In an embodiment, the % conversion of 2-oxo-4-(hydroxymethylphosphinyl)butyric acid to L-glufosinate, esters, or salts thereof is at least 1%.

In an embodiment, the process of the present invention is capable of converting 30% to 85% of 2-oxo-4-(hydroxymethylphosphinyl)butyric acid to L-glufosinate. In an embodiment, the process of the present invention is capable of converting more than 30% of 2-oxo-4-(hydroxymethylphosphinyl)butyric acid to L-glufosinate. In an embodiment, the process of the present invention is capable of converting more than 40% of 2-oxo-4-(hydroxymethylphosphinyl)butyric acid to L-glufosinate. In an embodiment, the process of the present invention is capable of converting more than 50% of 2-oxo-4-(hydroxymethylphosphinyl)butyric acid to L-glufosinate. In an embodiment, the process of the present invention is capable of converting more than 60% of 2-oxo-4-(hydroxymethylphosphinyl)butyric acid to L-glufosinate. In an embodiment, the process of the present invention is capable of converting more than 70% of 2-oxo-4-(hydroxymethylphosphinyl)butyric acid to L-glufosinate.

In an embodiment, the present invention provides L-glufosinate, esters, or salts thereof, wherein L-glufosinate, esters, or salts thereof is produced by a process using a biocatalyst derived from a micro-organism of Amycolatopsis sp.

In an embodiment, the present invention provides an agrochemical composition comprising L-glufosinate, esters, or salts thereof, wherein L-glufosinate, esters, or salts thereof is produced by a process using a biocatalyst derived from a micro-organism of Amycolatopsis sp.

In another embodiment, the agrochemical composition comprises L-glufosinate, esters, or salts thereof produced by a biocatalytic process; wherein the process comprises converting 2-oxo-4-(hydroxymethylphosphinyl)butyric acid to L-glufosinate, esters, or salts thereof, using a biocatalyst derived from a micro-organism of Amycolatopsis sp., in presence of an amine donor and optionally a cofactor, and optionally an agrochemically acceptable excipient.

In an embodiment, the present invention provides use of L-glufosinate, esters, or salts thereof; or an agrochemical composition comprising L-glufosinate, esters, or salts thereof, prepared according to the present process as described herein for controlling unwanted plants or weeds.

### Advantages of the Present Invention:

1. The present invention provides a “green” technique to obtain L-glufosinate, esters, or salts thereof.
2. The present invention provides a simple and efficient method to obtain L-glufosinate, esters, or salts thereof.
3. The bio-catalytic process of the present invention efficiently converts 2-oxo-4-(hydroxymethylphosphinyl)butyric acid to L-glufosinate, esters, or salts thereof, at a high substrate concentration.
4. The present invention provides L-glufosinate, esters, or salts thereof having high yields and enantiomeric excess (% ee).
5. The present invention provides a method to obtain L-glufosinate, esters, or salts thereof, using a biocatalyst derived from a micro-organism of Amycolatopsis sp., capable of producing enzyme(s) and cofactor.
6. The biocatalyst used in present invention can be reused or recycled multiple times without affecting the efficiency of the process.

### EXAMPLES

The present invention is more specifically explained by below examples. However, it should be understood that the scope of the present invention is not limited by the examples in any manner. It will be appreciated by any person skilled in this art that the present invention includes below examples and further can be modified and altered within the technical scope of the present invention.

### Analytical Method Details:

For detection profile of reactants and products, samples were analysed on high performance liquid chromatograph with UV detector using C-18 Column (Inert seal, ODS—35 μm, 4.6×250 mm).

For qualitative analysis of L-isomer And D-isomer of glufosinate, samples were analysed on high performance liquid chromatograph with UV detector using Chirex 3126 (D)-penicillamine LC column (150×4.6 mm).

### Example 1: Cultivation of Strain for the Biocatalytic Process

In the present study, for growth of biocatalyst, a culture of Amycolatopsis sp. (deposited as ATCC 39116) from glycerol stock was inoculated into a modified tryptone soy broth (5 ml) and incubated for 32 hours at about 37° C. Grown culture was then transferred to a 100 ml modified tryptone soy broth for 24 hours at about 37° C. Culture was inoculated at 2% v/v for scale up to desired cell quantity for biotransformation.

Cells grown were centrifuged @4000 rpm for 5 minutes and washed once with 0.1M Tris HCl pH 8 to remove excess media to get a biocatalyst having 85% moisture content. This biocatalyst derived from a micro-organism of Amycolatopsis sp. was used for preparation of L-glufosinate, esters, or salts thereof as represented in below examples.

### Example 2: Process for Preparation of L-Glufosinate

50 g (0.29 moles) of monosodium L-glutamate (in concentration of 500 g/L), 0.2 mM of pyridoxal 5′ phosphate and 20 g of the biocatalyst (in concentration of 200 g/L) obtained in Example 1 were added in a reaction vessel. 25 g (0.138 moles) of 2-oxo-4-(hydroxymethylphosphinyl)butyric acid (PPO) (in concentration of 250 g/L) was added to the reaction mixture in single lot. The pH of the reaction system was adjusted to about 8 using concentrated sodium hydroxide solution. The reaction system was then diluted to 100 ml volume with buffer solution (0.1M Tris HCl) and further maintained at about 8 pH using buffer solution (0.1M Tris HCl). The reaction was maintained at 30° C. with rotation speed at 200 rpm for 1 hour. The conversion of 2-oxo-4-(hydroxymethylphosphinyl)butyric acid (PPO) to L-glufosinate (LGF) was monitored using HPLC over a period of days and the reaction progress is as shown in Table 1.

TABLE 1

| No. of | PPO | LGF | % | % |
|---|---|---|---|---|
| Days | (g/L) | (g/L) | Assimilation | Conversion |
| 0 | 179.08 | 0.00 | 0.00 | 0.00 |
| 5 | 86.16 | 45.29 | 52.14 | 25.16 |
| 10 | 17.46 | 71.03 | 90.30 | 39.46 |
| 14 | 7.12 | 82.95 | 96.02 | 46.08 |

From above table, it was concluded that the biocatalyst used in the present invention, successfully converted 2-oxo-4-(hydroxymethylphosphinyl)butyric acid in concentration of 250 g/L to L-glufosinate (purity 72%) with total assimilation of 96.02% of 2-oxo-4-(hydroxymethylphosphinyl)butyric acid and 46.08% of conversion to L-glufosinate. The chiral ratio was about 99.60:0.4 (L:D).

### Example 3: Process for Preparation of L-Glufosinate by Reusing the Biocatalyst

The process according to Example 2 was carried out to prepare 10 ml reaction system having 25 g/L concentration of 2-oxo-4-(hydroxymethylphosphinyl)butyric acid, 0.2 mM of pyridoxal 5′ phosphate and 200 g/L concentration of whole-cell biocatalyst obtained in Example 1. Cycle 1 was performed to convert of 2-oxo-4-(hydroxymethylphosphinyl)butyric acid to L-glufosinate. After cycle 1, the biocatalyst was centrifuged, washed with water and reused for cycle 2. The same process for recycling of the biocatalyst was repeated after every cycle till completion of the process. The biocatalyst was recycled for 6 cycles and the conversion of 2-oxo-4-(hydroxymethylphosphinyl)butyric acid (PPO) to L-glufosinate (L-GF) was monitored using HPLC and the observation were listed in Table 2.

TABLE 2

| Cycle | Time | PPO | L-GF | % | % |
|---|---|---|---|---|---|
| No. | (hours) | (g/L) | (g/L) | Assimilation | Conversion |
| Cycle-1 | 0 | 23.36 | 0 | 0 | 0 |
| | 48 | 10.97 | 4.90 | 53 | 20.97 |
| | 96 | 2.09 | 6.98 | 91.05 | 29.88 |
| | 120 | 1.13 | 9.20 | 95.46 | 39.38 |
| Cycle-2 | 0 | 23.97 | 0 | 0 | 0 |
| | 48 | 8.34 | 6.48 | 65.20 | 23.03 |
| | 96 | 6.02 | 9.12 | 74.88 | 38.04 |
| | 120 | 4.25 | 10.22 | 83.02 | 42.63 |
| Cycle-3 | 0 | 22.73 | 0 | 0 | 0 |
| | 48 | 6.25 | 7.36 | 72.5 | 32.33 |
| | 96 | 1.36 | 10.02 | 94.01 | 44.08 |
| | 120 | 0.37 | 11.25 | 98.51 | 49.49 |
| Cycle-4 | 0 | 24.18 | 0 | 0 | 0 |
| | 48 | 4.26 | 9.36 | 82.20 | 39.90 |
| | 96 | 0.59 | 10.58 | 97.53 | 44.19 |
| | 120 | 0.00 | 12.6 | 99.08 | 52.63 |
| Cycle-5 | 0 | 23.59 | 0 | 0 | 0 |
| | 48 | 5.79 | 6.48 | 75.45 | 27.47 |
| | 96 | 3.06 | 8.31 | 87.03 | 35.22 |
| | 120 | 1.92 | 10.94 | 91.86 | 46.37 |
| Cycle-6 | 0 | 24.70 | 0 | 0 | 0 |
| | 48 | 9.49 | 4.74 | 61.57 | 19.19 |
| | 96 | 4.13 | 7.18 | 83.28 | 29.06 |
| | 120 | 2.34 | 10.35 | 90.52 | 41.90 |

From above observations, it can be concluded that the whole-cell catalyst used in the present invention can be used for preparing L-glufosinate, esters, or salts thereof, consequently for at least 6 cycles with consistent conversion of about 40% to about 50%.

### Example 4: Process for Preparation of L-Glufosinate without Cofactor

The process according to Example 2 was carried out, without pyridoxal 5′ phosphate to prepare 10 ml reaction system having 25 g/L concentration of 2-oxo-4-(hydroxymethylphosphinyl)butyric acid and 400 g/L concentration of the biocatalyst obtained in Example 1. The biocatalyst were reused for 5 cycles and the conversion of 2-oxo-4-(hydroxymethylphosphinyl)butyric acid (PPO) to L-glufosinate (L-GF) was monitored using HPLC and the observation were listed in Table 3.

TABLE 3

| | Time | PPO | L-GF | % | % |
|---|---|---|---|---|---|
| Cycle No. | (hours) | (g/L) | (g/L) | Assimilation | Conversion |
| Cycle-1 | 0 | 24.15 | 0.00 | 0.00 | 0.00 |
| | 72 | 6.72 | 7.38 | 72.15 | 30.55 |
| | 120 | 2.88 | 11.13 | 88.07 | 46.08 |
| Cycle-2 | 0 | 23.64 | 0.00 | 0.00 | 0.00 |
| | 72 | 4.72 | 9.14 | 80.08 | 38.66 |
| | 120 | 1.91 | 11.93 | 91.94 | 50.46 |
| Cycle-3 | 0 | 24.08 | 0.00 | 0.00 | 0.00 |
| | 72 | 2.39 | 8.71 | 90.07 | 36.17 |
| | 120 | 0.0 | 13.12 | 99.95 | 54.48 |
| Cycle-4 | 0 | 23.96 | 0.00 | 0.00 | 0.00 |
| | 72 | 5.24 | 7.62 | 78.13 | 31.8 |
| | 120 | 1.39 | 11.71 | 94.19 | 48.77 |
| Cycle 5 | 0 | 23.11 | 0.00 | 0.00 | 0.00 |
| | 72 | 6.54 | 8.83 | 71.68 | 38.20 |
| | 120 | 0.00 | 12.25 | 99.95 | 52.86 |

### Example 5: Process for Obtaining L-Glufosinate with Batch-Wise Addition of the Substrate

50 g (0.29 moles) of monosodium L-glutamate in concentration of 500 g/L, 0.2 mM of pyridoxal 5′ phosphate were added in the reaction flask and the biocatalyst obtained in Example 1 in concentration of 200 g/L was added to it. The pH of the reaction system was adjusted to about 8 using concentrated sodium hydroxide solution and the system was dilute to 65 ml volume with using buffer solution (0.1 M Tris HCl). The reaction was maintained at 30° C. with rotation speed at 200 rpm and 25 g (0.138 moles) of 2-oxo-4-(hydroxymethylphosphinyl)butyric acid (PPO) was added to the reaction mixture in 4 batches, each having concentration of 62.5 g/L (total PPO added is in concentration of 250 g/L) over the period of 48 hours at interval of 12 hours each. The conversion of 2-oxo-4-(hydroxymethylphosphinyl)butyric acid (PPO) to L-glufosinate (L-GF) was monitored using HPLC over a period of days and the reaction progress is as shown below Table 4.

TABLE 4

| No. of | PPO | L-GF | % | % |
|---|---|---|---|---|
| Days | (g/L) | (g/L) | Assimilation | Conversion |
| 0 | 0.00 | 0.00 | 0.00 | 0.00 |
| 0.5 | 20.34 | 2.88 | 44.97 | 1.98 |
| 5 | 99.01 | 46.04 | 44.99 | 31.68 |
| 10 | 49.87 | 69.27 | 72.29 | 47.67 |
| 14 | 34.68 | 75.75 | 80.74 | 52.11 |

From above table, it was concluded that when 2-oxo-4-(hydroxymethylphosphinyl)butyric acid was added in batches, 80.74% assimilation of 2-oxo-4-(hydroxymethylphosphinyl)butyric acid was obtained and the conversion to L-glufosinate was 52.11%.

### Example 6: Process for Preparation of L-Glufosinate Using Neutralised Substrate

7 g of (75% purity) PPO (0.28 M) was neutralised in a controlled manner using 20% ammonium carbonate solution till pH 8 in the ice bath. In this solution, 8 g of L-glutamic acid (0.54 M), 0.2 mM of pyridoxal 5′ phosphate and 20 g of biocatalyst was added. The pH of the reaction system was again adjusted to 8 using 20% ammonium carbonate solution. The reaction was maintained at 40° C. with rotation speed at 300 rpm for 90 hours. The conversion of 2-oxo-4-(hydroxymethylphosphinyl)butyric acid (PPO) to L-glufosinate (L-GF) was monitored using HPLC over a period of days and the reaction progress is as shown in the Table 5.

TABLE 5

| Time | PPO | L-GF | % | % |
|---|---|---|---|---|
| (hours) | (g/L) | (g/L) | Assimilation | Conversion |
| 0 | 51.00 | 0.00 | 0.00 | 0.00 |
| 48 | 15.70 | 28.90 | 69.22 | 56.67 |
| 72 | 7.20 | 36.50 | 85.88 | 71.57 |
| 96 | 4.50 | 39.50 | 91.18 | 77.45 |

From above table, it was observed that biocatalyst used in the present invention, successfully converted 2-oxo-4-(hydroxymethylphosphinyl)butyric acid (purity 75%) in concentration of 50 g/L to L-glufosinate with total assimilation of 91.18% of 2-oxo-4-(hydroxymethylphosphinyl)butyric acid and 77.45% of conversion to L-glufosinate. The chiral ratio was about 99.68:0.32 (L:D).

1. A process for preparing L-glufosinate, esters, or salts thereof, the process comprising:
converting 2-oxo-4-(hydroxymethylphosphinyl)butyric acid to L-glufosinate, esters, or salts thereof using a biocatalyst derived from a micro-organism of Amycolatopsis sp.

2. The process as claimed in claim 1, wherein the micro-organism of Amycolatopsis sp. is deposited as ATCC 39116.

3. The process as claimed in claim 1, wherein the biocatalyst is selected from isolated enzymes; partially purified enzymes; cell-free extract or crude cell extract liquid, powder, or immobilized/fixed form; permeabilized cells; whole cells; whole fermentation broths; lyophilized cells; or combinations thereof.

4. The process as claimed in claim 3, wherein the biocatalyst comprises whole cells, whole fermentation broths, permeabilized cells, or lyophilized cells.

5. The process as claimed in claim 4, wherein the biocatalyst comprises one or more enzymes selected from amino-transferases, amino-acid dehydrogenases, and aldo-keto reductases.

6. The process as claimed in claim 1, wherein the process is carried out in the presence of an amine donor.

7. The process as claimed in claim 6, wherein the amine donor is selected from L-aspartate or racemic aspartate or salts thereof, L-glutamate or racemic glutamate or salts thereof, L-alanine or racemic alanine or salts thereof, L-phenylalanine or racemic phenylalanine or salts thereof, L-glycine or racemic glycine or salts thereof, L-lysine or racemic lysine or salts thereof, L-valine or racemic valine or salts thereof, L-serine or racemic serine or salts thereof, L-glutamine or racemic glutamine or salts thereof; inorganic ammonia source, ammonia; organic amine such as isopropylamine, sec-butylamine, ethanolamine, 2-aminobutyric acid or salts thereof, and diaminoproprionic acid or salts thereof.

8. The process as claimed in claim 1, wherein the process is carried out in the presence of a cofactor.

9. The process as claimed in claim 8, wherein the cofactor is either added externally or produced by Amycolatopsis sp.

10. The process as claimed in claim 9, wherein the cofactor is pyridoxal 5′ phosphate.

11. The process as claimed in claim 1, wherein the process is carried out at a pH ranging from about 6 to about 9.

12. The process as claimed in claim 1, wherein e biocatalyst is recycled at least about 5 times.

13. The process as claimed in claim 1, wherein the process optionally comprises batchwise addition of the 2-oxo-4-(hydroxymethylphosphinyl)butyric acid.

14. The process as claimed in claim 1, wherein a % assimilation of 2-oxo-4-(hydroxymethylphosphinyl)butyric acid is at least 30%.

15. The process as claimed in claim 1, wherein a % conversion of 2-oxo-4-(hydroxymethylphosphinyl)butyric acid to L-glufosinate, esters, or salts thereof is at least 1%.

16. L-glufosinate, esters, or salts thereof prepared by the process as claimed in claim 1.

17. An agrochemical composition comprising L-glufosinate, esters, or salts thereof, wherein L-glufosinate, esters, or salts thereof is prepared by the process as claimed in claim 1.
